(19) United States
(12) Patent Application Publication
(10) Pub. No.: US 2026/0068978 A1
(43) Pub. Date: Mar. 12, 2026

(54) SYSTEMS AND METHODS FOR REPLACING STRAPS FOR HELMET SUSPENSION

(71) Applicant: Welshco LLC, Lawton, OK (US)

(72) Inventor: Geoffrey Welsh, Lawton, OK (US)

(21) Appl. No.: 19/389,479

(22) Filed: Nov. 14, 2025

Related U.S. Application Data
(63) Continuation-in-part of application No. 19/218,801, filed on May 27, 2025.
Application No. 19/218,801 is a continuation of application No. 18/102,194, filed on Jan. 27, 2023, now Pat. No. 12,329,228.
(60) Provisional application No. 63/304,752, filed on Jan. 31, 2022.

(51) Int. Cl. A42B 3/08; A42B 3/14; A61F 9/06

(52) U.S. Cl. CPC A42B 3/08; A42B 3/145; A61F 9/06

(57) ABSTRACT

A system and method for replacing the rear and/or top strap of a helmet suspension assembly is disclosed. The system includes a pair of adapter rings, each configured to fit over an attachment boss of a helmet's headgear assembly. Replacement straps, which may be made of stretchable or reinforced materials, are attached to the adapter rings and extend between them to provide secure and adjustable support. The adapter rings and straps can be installed on existing helmet assemblies by removing the original straps and fitting the adapter rings over the attachment bosses. The invention accommodates various boss shapes and provides improved comfort, durability, and ease of replacement for helmet users.

### CROSS REFERENCE TO RELATED APPLICATIONS

This application is a continuation-in-part of U.S. Non-Provisional application Ser. No. 19/218,801, filed May 27, 2025, titled “Method for Replacing a Strap for Helmet Suspension,” which is a continuation of U.S. Non-Provisional application Ser. No. 18/103,194, filed Jan. 27, 2023, titled “Replacement Strap for Helmet Suspension,” which issued as U.S. Pat. No. 12,329,228 on Jun. 4, 2025, and claims the benefit of U.S. Provisional Patent Application No. 63/304,752 , filed Jan. 31, 2021. The disclosures of these applications and the issued patent are incorporated herein by reference in their entirety.

### FIELD OF THE INVENTION

This invention relates generally to the field of protective helmets and more particularly, but not by way of limitation, to systems and methods for replacing a rear and top strap of a helmet for a helmet suspension.

### BACKGROUND

A wide variety of helmets are used for safety purposes in industrial and recreational activities. Hard hats and welding helmets are commonly worn to protect workers from impact, sparks, and other workplace hazards. For example, FIG. 1 depicts a PRIOR ART welding helmet 200. The welding helmet 200 includes a face shield 202, a lens 204, and a standard headgear assembly 206. The face shield 202 protects the welder's face from sparks, slag and debris produced during the welding process. The lens 204 is permanently or temporarily tinted to reduce the high intensity light from harming the welder's eyes. The standard headgear assembly 206 supports the helmet 200 on the head of the welder. In some cases, the standard headgear assembly 206 is referred to as a helmet “suspension” because the helmet 200 rides on the standard headgear assembly 206, which is in turn connected to the welder's head.

The PRIOR ART standard headgear assembly 206 is depicted in isolation in FIG. 2. The standard headgear assembly 206 generally includes a front strap 208, a standard rear strap 210, a standard top strap 212, and two hubs 214. In the embodiment depicted in FIGS. 1-2, the front strap 208, standard rear strap 210 and standard top strap 212 each extend between and are joined together at the two hubs 214.

The front strap 208 may optionally include a cloth sweatband 216 designed to absorb perspiration from the welder's forehead. The standard rear strap 210 includes a rear strap adjustment mechanism 218 that is configured to increase or decrease the length of the standard rear strap 210, which enables the standard headgear assembly 206 to be tightened around the back of the welder's head. The internal circumference created by the front strap 208, the standard rear strap 210 and the connecting hubs 214 can be adjusted to match the circumference of the welder's head. In the embodiment depicted in FIGS. 1-2, the rear strap adjustment mechanism 218 includes a ratcheting system in which a dial is rotated to increase or decrease the length of the standard rear strap 210. For heavier helmets 200, it is particularly important for the standard rear strap 210 to be tightened about the welder's head so that the helmet 200 remains securely fastened during use.

The standard top strap 212 includes a top strap adjustment mechanism 220. In the embodiment depicted in FIGS. 1-2, the top strap adjustment mechanism 220 includes a series of adjustment holes and posts that cooperate to adjust the length of the standard top strap 212. In this way, the length of the top strap 212 can be adjusted so that the front strap 208 and standard rear strap 210 are correctly positioned on the welder's head.

The face shield 202 is connected to the standard headgear assembly 206 with fasteners 222. Each fastener 222 includes a threaded bolt 224 with an attachment boss 230 having a bolt base 226, and a securing ring 228 that is configured to be hand-tightened onto the threaded bolt 224 over the attachment boss 230. The attachment boss 230 is designed to contact the inside of the sides of the face shield 202. Each attachment boss 230 includes a fastener bore 232, which is configured to receive the threaded bolt 224. The bolt base 226 is preferably larger than the fastener bore 232. Each attachment boss 230 also has an outer perimeter defined by side surfaces 234.

As best shown in FIG. 2, the attachment bosses 230 extend outward from the hubs 214 such that the bolt base 226 is retained within the interior of the corresponding attachment boss 230 and not in contact with the welder's head. Each of the threaded bolts 224 extends through the corresponding fastener bore 232 and through a mating hole in the face shield 202, where the securing ring 228 is secured to the threaded bolt 224 on the outside of the face shield 202 and the outside of the attachment boss. Tightening the securing ring 228 onto the threaded bolt 224 applies a compressive force between the attachment boss 230 and face shield 202.

In most helmets 200, the front strap 208, standard rear strap 210 and standard top strap 212 are manufactured from a thermoplastic through an injection molding process. In this way, the front strap 208, standard rear strap 210, standard top strap 212, and hubs 214 can be constructed as a unitary piece that is assembled by connecting two ends of the standard rear strap 210 at the rear strap adjustment mechanism 218 (which may be separately manufactured) and connecting two ends of the standard top strap 212 at the top strap adjustment mechanism 220 (which may also be separately manufactured).

Many helmets 200 are designed such that the face shield 202 can be pivoted from a deployed (lowered) position to protect the welder's face while welding to a retracted (raised) position that permits the welder to see under the raised helmet while preparing for a welding operation. To support the weight of the face shield 202 in the raised position, the nuts 228 must be sufficiently tightened to apply an appropriate amount of compression and friction between attachment bosses 230 and the inside of the face shield 202.

Although the basic form and function of the standard headgear assembly 206 depicted in FIGS. 1 and 2 has found widespread acceptance, the standard headgear assembly 206 nonetheless suffers from several deficiencies. In particular, the rear strap adjustment mechanism 218 and the top strap adjustment mechanism 220 are each prone to failure after extended use. Because the rear strap adjustment mechanism 218 and top strap adjustment mechanism 220 are often tightened or loosened each time the helmet 200 is donned or doffed by the welder, the rear strap adjustment mechanism 218 and top strap adjustment mechanism 220 often fail before the balance of the components within the helmet 200. In some cases, the standard rear strap 210 and standard top strap 212 are uncomfortable, particularly when worn for extended periods. There is, therefore, a need for an improved headgear assembly for use in a hardhat, welding helmet or other helmet that includes a helmet suspension.

### SUMMARY OF THE INVENTION

In one aspect, embodiments of the present disclosure are directed to a replacement strap assembly for use on a helmet, where the helmet has a headgear assembly that includes a front strap, a pair of hubs, and a pair of attachment bosses extending from the pair of hubs. The replacement strap assembly comprises a pair of adapter rings, each having an adapter ring aperture that is sized and configured to be placed over a corresponding attachment boss. The replacement strap assembly further includes a replacement rear strap extending between a rear side of the pair of adapter rings and, in certain embodiments, a replacement top strap extending between a top side of the pair of adapter rings. The adapter ring apertures and attachment bosses may be D-shaped, circle-shaped, or otherwise configured to match the geometry of the headgear assembly. In some embodiments, the replacement rear strap and/or replacement top strap are formed from stretchable fabric or reinforced materials, and may be secured using stitching, Velcro, clips, or other fasteners.

The replacement strap assembly may be installed by removing the original rear and/or top strap(s) from the headgear assembly and fitting the adapter rings over the attachment bosses, thereby providing an inexpensive, durable, and comfortable modification or replacement for conventional helmet suspension straps. In certain embodiments, the replacement strap assembly may be used in addition to, or without removing, the original straps, and may be adapted for use with a variety of helmet types and attachment boss configurations.

### BRIEF DESCRIPTION OF THE DRAWINGS

FIG. 1 provides a partial cutaway depiction of a PRIOR ART welding helmet with headgear assembly.

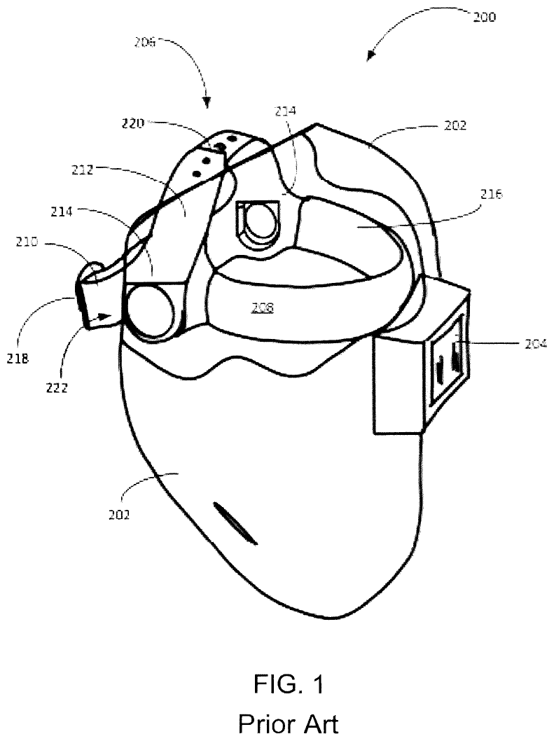

FIG. 2 is a perspective view of the PRIOR ART headgear assembly of FIG. 1.

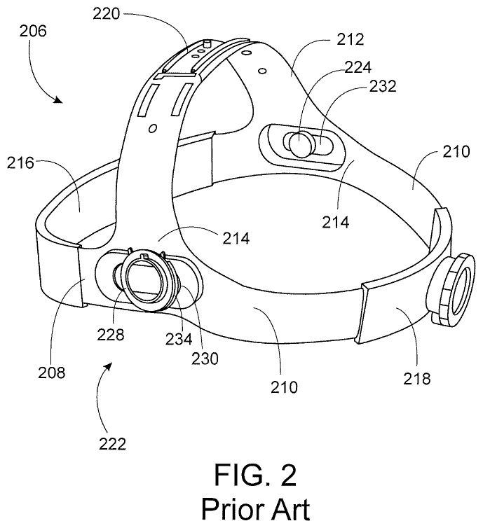

FIG. 3 depicts a portion of the headgear assembly modified in accordance with an exemplary embodiment of the present invention.

FIG. 4 depicts an adapter ring and replacement rear strap of the replacement rear strap assembly.

FIG. 5 depicts the replacement rear strap assembly of FIG. 5 connected to the modified headgear assembly of FIG. 3.

FIG. 6 depicts two adapter rings and a strap clip in accordance with an exemplary embodiment of the present invention.

FIG. 7 depicts an exploded view of the headgear assembly with an adapter ring in accordance with an exemplary embodiment of the present invention.

FIG. 8 provides a rear perspective view of the replacement strap assembly and modified headgear assembly.

FIG. 9 depicts two adapter rings with a strap in accordance with an exemplary embodiment of the present invention.

FIG. 10 depicts a portion of the headgear assembly modified in accordance with an exemplary embodiment of the present invention.

FIG. 11 depicts two adapter rings and a strap clip in accordance with an exemplary embodiment of the present invention.

FIG. 12 depicts an adapter ring, replacement rear strap, and replacement top strap of the replacement strap assembly.

FIG. 13 depicts the replacement strap assembly of FIG. 12 connected to the modified headgear assembly of FIG. 10.

FIG. 14 depicts a perspective view of the modified headgear assembly in accordance with an exemplary embodiment of the present invention.

FIG. 15 depicts two adapter rings with a replacement rear strap and replacement top strap in accordance with an exemplary embodiment of the present invention.

### DETAILED DESCRIPTION

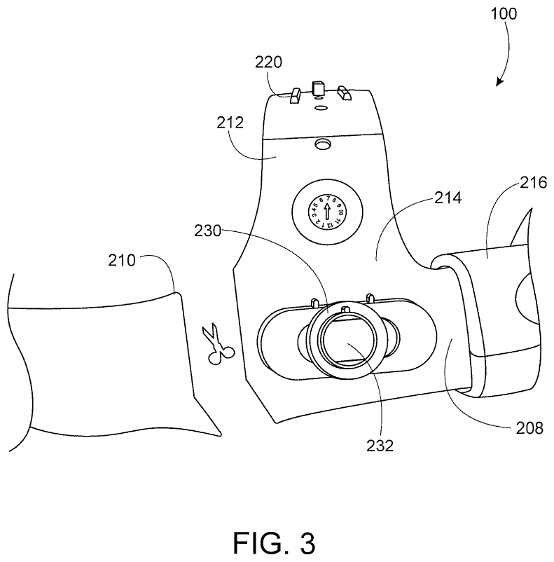

In accordance with an exemplary embodiment, FIG. 3 depicts a modified headgear assembly 100. The modified headgear assembly 100 includes the front strap 208, standard top strap 212, hubs 214 and attachment bosses 230 common to the prior art standard headgear assembly 206, but the standard rear strap 210 and rear strap adjustment mechanism 218 have been removed. In some embodiments, the standard rear strap 210 can be removed simply by severing the standard rear strap 210 from both hubs 214. Scissors, utility knives or other cutting tools can be used to remove the standard rear strap 210 from the hubs 214. In other embodiments, a standard headgear assembly 206 may be modified during initial manufacture to omit the standard rear strap 210.

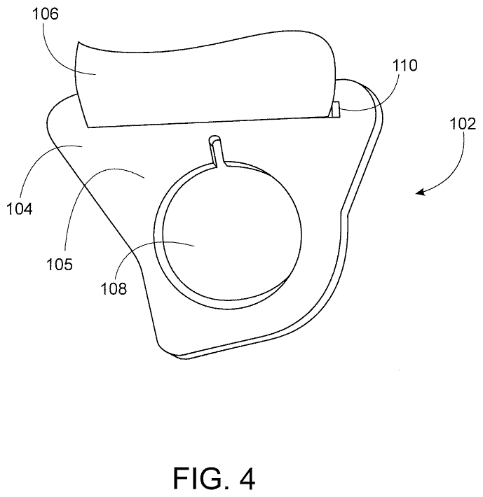

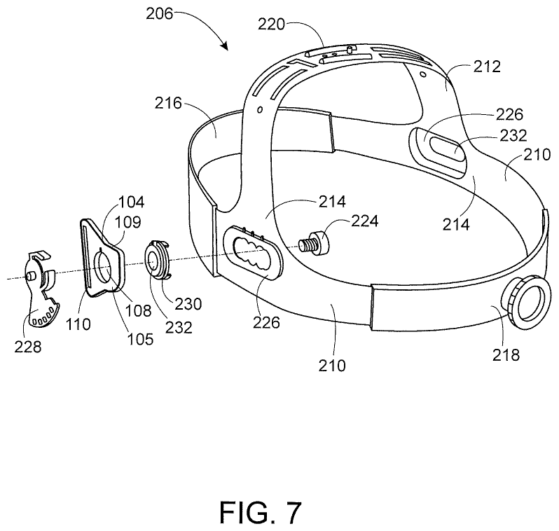

FIGS. 4-9 depict a replacement rear strap assembly 102. The replacement rear strap assembly 102 includes a pair of adapter rings 104 and a replacement rear strap 106 that extends between the pair of adapter rings 104. In some embodiments, a 3-D printing process is used to create each adapter ring 104 from any material suitable for a 3-D printer. In a preferred embodiment using a 3-D printing process, each adapter ring 104 is made from polycarbonate. It will be appreciated, however, that each adapter ring 104 may be made by any other suitable manufacturing processes and materials. Further, each adapter ring 104 may be made through the combination of one or more processes and from one or more materials. For example, each adapter ring 104 may be made from any suitable plastic, metal, wood, fabric or textile or any combination thereof.

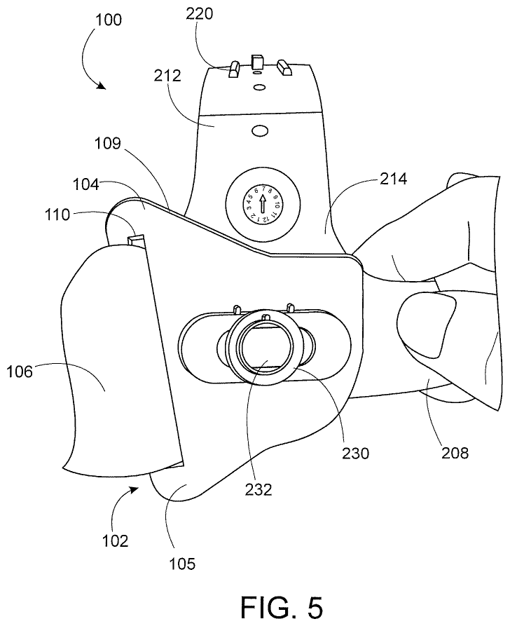

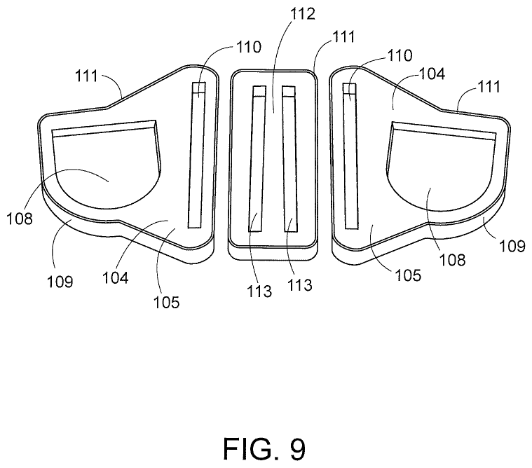

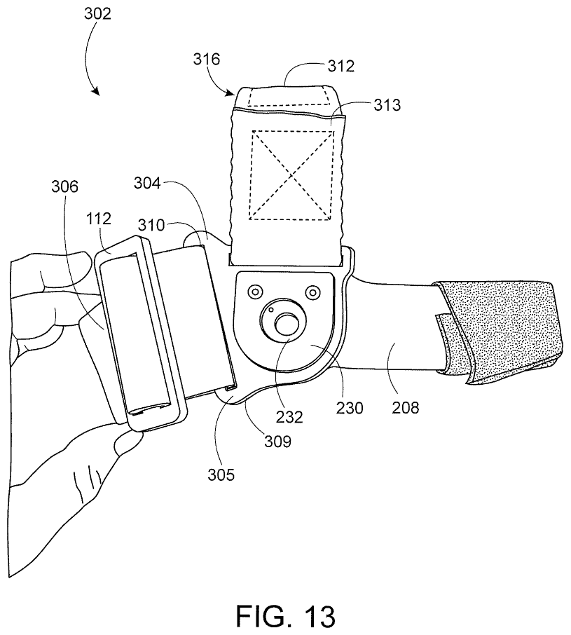

Each adapter ring 104 includes an adapter ring aperture 108 that is designed to fit around a corresponding one of the attachment bosses 230. As illustrated in FIGS. 3 and 5, the attachment boss 230 is “circle-shaped” and as illustrated in FIGS. 4, 6, and 7, the adapter ring aperture 108 is congruently “circle-shaped” and sized to closely fit over the attachment boss 230, such that the adapter ring aperture 108 corresponds to the outer perimeter of the attachment boss 230. It will be appreciated, however, that the adapter ring aperture 108 can be configured in any shape to match or accommodate the outer perimeter of a corresponding attachment boss 230, which may take on a variety of shapes and configurations. For example, as depicted in FIGS. 9-15, the adapter ring aperture 308 is “D-shaped” to match the corresponding attachment boss 230, which is also “D-shaped.”

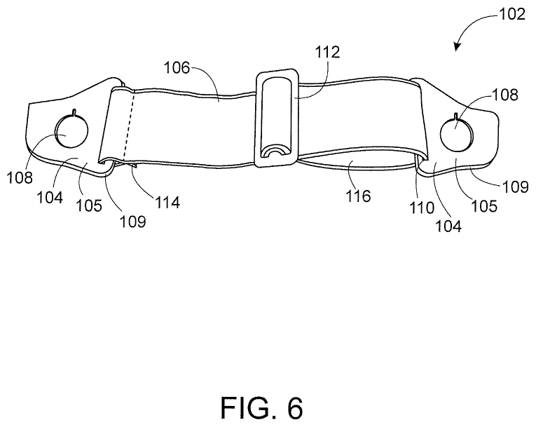

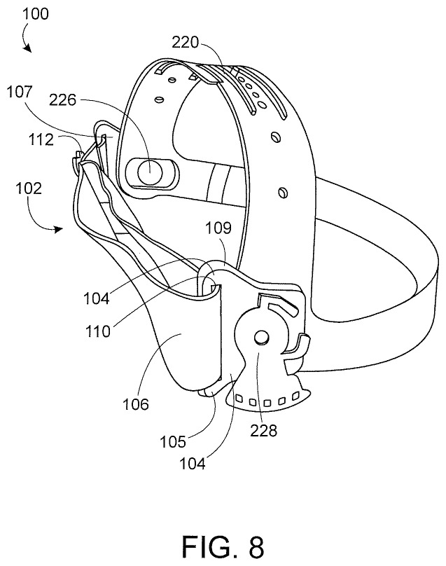

Each adapter ring 104 further includes an upper surface 105, a lower surface 107 positioned opposite of the upper surface 105 (depicted in FIG. 8), and outer side surfaces 109 that extend from edges of the upper surface 105 to corresponding edges of the lower surface 107 and define an outer perimeter of the adapter ring 104. As shown in FIG. 9, the upper surface 105 may include a fillet 111 at its edges. In some embodiments, the fillet 111 is between 1 mm to 2 mm in width. In several embodiments, the fillet 111 is 1.2 mm in width. The outer perimeter of each adapter ring 104 may be a streamlined shape with obtuse angles, curves, and straight edges. Notwithstanding these exemplary embodiments, it will be appreciated that the outer perimeter of each adapter ring 104 can be configured in any shape or size to accommodate the adapter ring aperture 108. In some embodiments, the outer perimeter may include sharp angles rather than curves. As illustrated in FIG. 6, each adapter ring 104 may have a shape and size mirroring that of the other. However, it is appreciated that the adapter rings 104 in some embodiments can have a different shape and size from each other.

As shown in FIG. 9, each of the adapter rings 104 can include a strap slit 110 that is sized to permit the replacement rear strap 106 to be passed through the strap slit 110 (as depicted in FIG. 6). In some embodiments, the strap slit 110 has a height that the same as the width of the replacement rear strap 106. In other embodiments, the strap slit 110 has a height that is greater than the width of the replacement rear strap 106. In a first preferred embodiment, the replacement rear strap 106 has a width of 2 inches. In a second preferred embodiment, the replacement rear strap 106 has a width of 1.5 inches. The replacement rear strap 106 of FIG. 8 may be manufactured to any width and length that is suitable for securely fastening the modified headgear assembly 100 to a user's head during use.

As illustrated in FIG. 6, the replacement rear strap 106 has a short end 114 that passes through the strap slit 110 of a first adapter ring 104 and is then attached by stitching or by other suitable attachment devices, such as adhesive, snaps, buttons, clips or other closure mechanisms, to a first position on the replacement strap 106 that is proximate to the first adapter ring 104. The replacement rear strap 106 of FIG. 6 also has a long end 116 that passes through the strap slit 110 of a second adapter ring 104 and is then attached by stitching or by other suitable attachment devices to a second position on the replacement rear strap 106 that is distant from the second adapter ring 104.

The strap clip 112 of the replacement rear strap 106 permits the replacement rear strap 106 to be lengthened or shortened. It is understood, however, that other mechanisms may be used to permit the replacement strap 106 to be lengthened or shortened, such as a hook and loop fastener, a double ring closure, Velcro, snaps, or other known mechanisms.

In some of the embodiments, a 3-D printing process is used to create the strap clip 112 from any material suitable for a 3-D printer. In a preferred embodiment using a 3-D printing process, the strap clip 112 is made from polycarbonate. It will be appreciated, however, that the strap clip 112 may be made by any other suitable manufacturing processes and materials and, in fact, may be made through the combination of one or more processes and from one or more materials. For example, the strap clip 112 may be made from any suitable plastic, metal, wood, fabric or textile or any combination thereof. As shown in FIG. 9, the strap clip 112 may have two strap apertures 113 that are sized to permit the replacement rear strap 106 to be passed through the strap apertures 113. In other embodiments, the strap clip 112 may instead have only one strap aperture 113. In several embodiments, the replacement rear strap 106 may have multiple strap clips 112. For example, the replacement rear strap 106 may have two strap clips 112, each strap clip 112 having only one strap aperture 113. The strap clip 112 may also include a fillet 111 at its edges. In some embodiments, the fillet 111 is between 1 mm to 2 mm in width. In several embodiments, the fillet 111 is 1.2 mm in width.

In some embodiments, the replacement rear strap 106 is manufactured from an elastic material that stretches in one or more directions. Suitable strap material is commonly found in the straps used on standard ski goggles and headlamps. In some embodiments, the replacement rear strap 106 may include a non-slip silicone strip. In several embodiments, an additional reinforcing material, such as leather or rubber, may be used to provide more structure to the replacement rear strap 106. The replacement rear strap 106 may be patterned, colored, or textured to alter the visual appearance.

As illustrated in FIGS. 3-9, the replacement rear strap assembly 102 is attached to the modified headgear assembly 100 by removing the standard headgear assembly 206 from the helmet 200, removing the standard rear strap 210 from the standard headgear assembly 206 to provide the modified headgear assembly 100, securing the replacement rear strap assembly 102 to the modified headgear assembly 100 by placing each of the apter rings 104 over a corresponding one of the attachment bosses 230, and then attaching the modified headgear assembly 100 to the face shield 202 with the fasteners 222. In this way, the adapter rings 104 are captured on the attachment bosses 230 between the hubs 214 and the interior of the face shield 202.

The attachment bosses 230 on worn-out or older headgear may develop rough edges that make it difficult to slide hardware over the outer perimeter of the attachment bosses 230. Therefore, depending on the condition of the standard headgear assembly, it may be necessary to sand or file down these rough edges on the attachment bosses 230 before placing each of the adapter rings 104 over a corresponding one of the attachment bosses 230. It will be appreciated that the fasteners 222 may be made from any suitable material, including but not limited to metal, plastic, or wood.

In most embodiments, the outer side surfaces 109 of the adapter rings 104 have approximately the same width as the side surfaces 234 of the attachment bosses 230, such that the adapter rings 104 are captured tightly between the hubs 214 and the interior of the face shield 202 during use. In other embodiments, the outer side surfaces 109 of the adapter rings 104 have a smaller width than the side surfaces 234 of the attachment bosses 230, such that the adapter rings are more loosely captured between the hubs 214 and the interior of the face shield 202. The replacement rear strap assembly 102 provides an inexpensive, durable, and comfortable modification to the standard rear strap 210 found in conventional headgear assemblies.

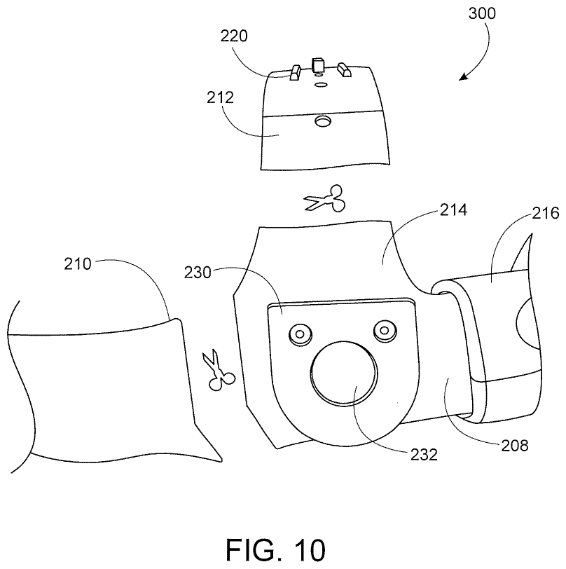

Now turning to FIGS. 10-15, in accordance with an exemplary embodiment, FIG. 10 depicts a modified headgear assembly 300. The modified headgear assembly 300 includes the front strap 208, hubs 214 and attachment bosses 230 common to the prior art standard headgear assembly 206, but the standard rear strap 210, rear strap adjustment mechanism 218, standard top strap 212 and top strap adjustment mechanism 220 have been removed. In some embodiments, the standard rear strap 210 and standard top strap 212 can both be removed simply by severing the standard rear strap 210 and standard top strap 212 from both hubs 214. Scissors, utility knives or other cutting tools can be used to remove the standard rear strap 210 and standard top strap from the hubs 214. It will be understood that only the standard top strap 212 could be replaced, or as already described, only the standard rear strap 210 could be replaced. In other embodiments, a standard headgear assembly 206 may be modified during initial manufacture to omit the standard rear strap 210 and/or the standard top strap 212.

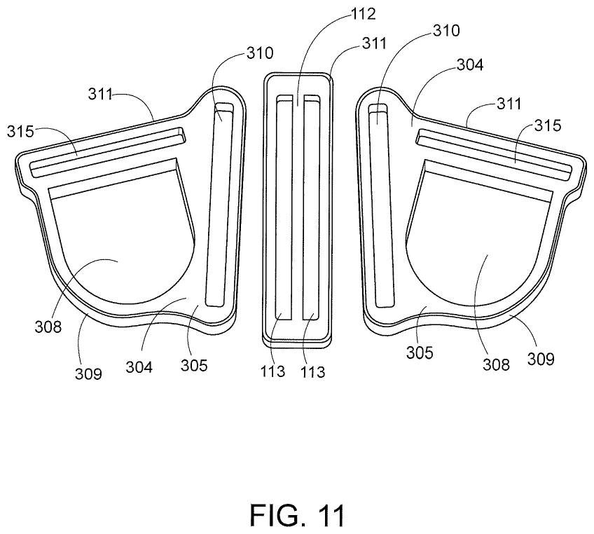

As depicted in FIG. 11-15, the second modified headgear assembly 300 includes a replacement strap assembly 302 and a pair of adapter rings 304. In some embodiments, a 3-D printing process is used to create each adapter ring 304 from any material suitable for a 3-D printer. In a preferred embodiment using a 3-D printing process, each adapter ring 304 is made from polycarbonate. It will be appreciated, however, that each adapter ring 304 may be made by any other suitable manufacturing processes and materials. Further, each adapter ring 304 may be made through the combination of one or more processes and from one or more materials. For example, each adapter ring 304 may be made from any suitable plastic, metal, wood, fabric or textile or any combination thereof.

Each adapter ring 304 includes a first strap slit 310 to allow the replacement rear strap 106 to be passed through the first strap slit 310, and a second strap slit 315 to permit the replacement top strap 316 to be passed through the second strap slit 315. In some embodiments, the first strap slit 310 and the second strap slit 315 each have a height that is the same as the width of each of the replacement top strap 316 and the replacement rear strap 106. In other embodiments, the first strap slit 310 and the second strap slit 315 have a height that is greater than the width of the rear replacement rear strap 106 and the replacement top strap 316. In a first preferred embodiment, each replacement strap 106 and 316 have a width of 2 inches. In a second preferred embodiment, each replacement strap 106 and 316 each have a width of 1.5 inches. The replacement rear strap 106 and replacement top strap 316 of FIGS. 11-15 may be manufactured to any width and length that is suitable for securely fastening the modified headgear assembly 300 to a user's head during use.

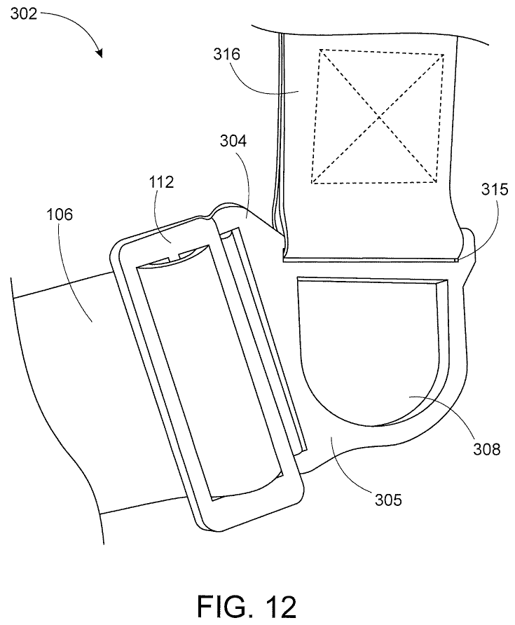

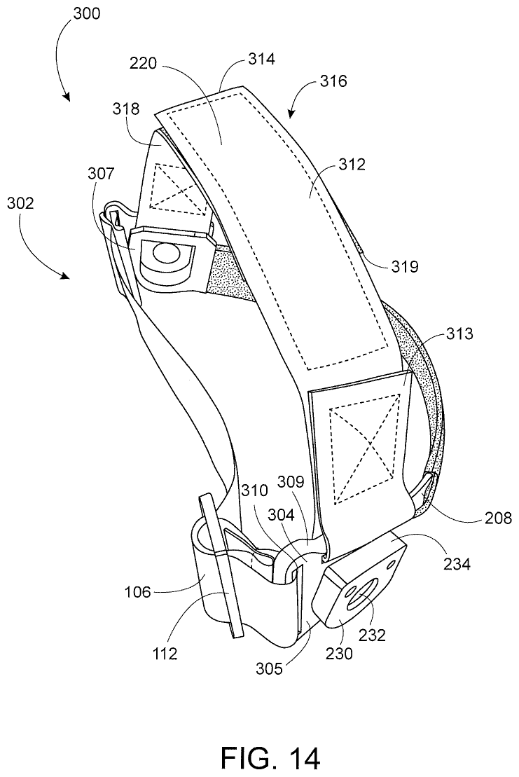

As depicted in FIG. 12, each adapter ring 304 includes an adapter ring aperture 308 that is designed to fit around a corresponding one of the attachment bosses 230. Each adapter ring 304 further includes an upper surface 305, a lower surface 307 (depicted in FIG. 14) positioned opposite of the upper surface 305, and outer side surfaces 309 that extend from edges of the upper surface 305 to corresponding edges of the lower surface 307 and define an outer perimeter of the adapter ring 304. As shown in FIG. 11, the upper surface 305 may include a fillet 311 at its edges. In some embodiments, the fillet 311 is between 1 mm to 2 mm in width. In several embodiments, the fillet 311 is 1.2 mm in width.

The outer perimeter of each adapter ring 304 may be a streamlined shape with obtuse angles, curves, and straight edges. Notwithstanding these exemplary embodiments, it will be appreciated that the outer perimeter of each adapter ring 304 can be configured in any shape or size to accommodate the adapter ring aperture 308. In some embodiments, the outer perimeter may include sharp angles rather than curves. As illustrated in FIGS. 11-15, each adapter ring 304 may have a shape and size mirroring that of the other. However, it is appreciated that the adapter rings 304 in some embodiments can have a different shape and size from each other.

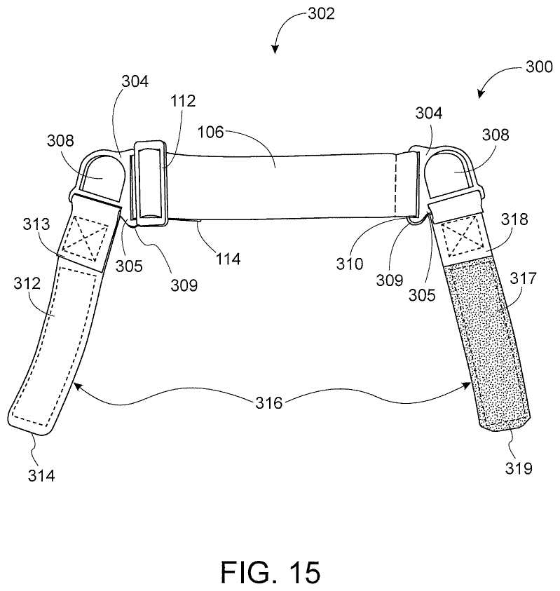

As depicted in FIG. 15, the replacement top strap 316 has a first side 312 with a first end 313 and a second end 314 and a second side 317 with a first end 318 and a second end 319. The first end 313 of the first side 312 passes through the strap slit 315 of a first adapter ring 304 and is then secured by stitching or by other suitable attachment devices, such as adhesive, snaps, buttons, clips or other closure mechanisms, to a first position on the first side 312 that is proximate to the first adapter ring 304. The first end 318 of the second side 317 passes through the strap slit 315 of a second adapter ring 304. and is then secured by stitching or by other suitable attachment devices, such as adhesive, snaps, buttons, clips or other closure mechanisms, to a second position on the second side 317 that is proximate to the second adapter ring 304. The first side 312 and the second side 317 of the replacement top strap 316 preferably are configured with opposing Velcro sides to allow them to be secured together to form the replacement stop strap 316. And allow them to be tightened on a user's head. It is understood, however, that other mechanisms may be used to permit the replacement top strap 316 to be lengthened or shortened, such as a clip (similar to the disclosed replacement rear strap 106), a hook and loop fastener, or a double ring closure to allow the strap to be tightened or lengthened as needed.

In some embodiments, the replacement top strap 316 is manufactured from an elastic material that stretches in one or more directions. Suitable strap material is commonly found in the straps used on standard ski goggles, headlamps, and for securing cables, equipment, and gear. In some embodiments, the replacement top strap 316 may include a non-slip silicone strip. In several embodiments, an additional reinforcing material, such as leather or rubber, may be used to provide more structure to the replacement top strap 316. The replacement top strap 316 may be patterned, colored, or textured to alter the visual appearance.

As illustrated in FIGS. 10-15, the replacement strap assembly 302 can be attached to the modified headgear assembly 300 by removing the standard headgear assembly 206 from the helmet 200, removing the standard rear strap 210 and the standard top strap 212 from the standard headgear assembly 206 to provide the modified headgear assembly 300, securing the replacement rear strap assembly 102 and the replacement top strap assembly 302 to the modified headgear assembly 300 by placing each of the adapter rings 304 over a corresponding one of the attachment bosses 230, and then attaching the modified headgear assembly 300 to the face shield 202 with the fasteners 222. In this way, the adapter rings 304 are captured on the attachment bosses 230 between the hubs 214 and the interior of the face shield 202. The attachment bosses 230 on worn-out or older headgear may develop rough edges that make it difficult to slide hardware over the outer perimeter of the attachment bosses 230. Therefore, depending on the condition of the standard headgear assembly, it may be necessary to sand or file down these rough edges on the attachment bosses 230 before placing each of the adapter rings 304 over a corresponding one of the attachment bosses 230. It will be appreciated that the fasteners 222 may be made from any suitable material, including but not limited to metal, plastic, or wood. In most embodiments, the outer side surfaces 309 of the adapter rings 304 have approximately the same width as the side surfaces 234 of the attachment bosses 230, such that the adapter rings 304 are captured tightly between the hubs 214 and the interior of the face shield 202 during use. In other embodiments, the outer side surfaces 309 of the adapter rings 304 have a smaller width than the side surfaces 234 of the attachment bosses 230, such that the adapter rings are more loosely captured between the hubs 214 and the interior of the face shield 202. The replacement rear strap assembly 102 and replacement top strap assembly 302 provide an inexpensive, durable, and comfortable modification to the standard rear strap 210 and standard top strap 212 found in conventional headgear assemblies.

Although the use of the replacement strap assemblies 102 and 302 have been illustrated in connection with the welding helmet 200, it will be understood that the replacement strap assemblies 102 and 302 can also be used on other hats and helmets, including, but not limited to, hard hats, climbing helmets, ski helmets, bicycle helmets, and motorcycle helmets. Additionally, although the replacement strap assemblies 102 and 302 are designed to replace the standard rear strap 210 and/or standard top strap 212 of the standard headgear assembly 206, it will be appreciated that the replacement strap assemblies 102 and/or 302 can be used in addition to, and without removing, the standard rear strap 210 or standard top strap 212. In some embodiments, the standard headgear assembly 206 or modified headgear assembly 100 or modified headgear assembly 300 may not include attachment bosses 230. For those applications, the adapter rings 104 or 304 can otherwise be connected to the modified headgear assembly 100, modified headgear assembly 300, or standard headgear assembly 206 using the fasteners 222 and an appropriate adapter ring aperture 308.

It is to be understood that even though numerous characteristics and advantages of various embodiments of the present invention have been set forth in the foregoing description, together with details of the structure and functions of various embodiments of the invention, this disclosure is illustrative only, and changes may be made in detail, especially in matters of structure and arrangement of parts within the principles of the present invention to the full extent indicated by the broad general meaning of the terms in which the appended claims are expressed. It will be appreciated by those skilled in the art that the teachings of the present invention can be applied to other systems without departing from the scope and spirit of the present invention.

What is claimed is:

1. A helmet comprising:
a headgear assembly comprising:
a front strap;
a pair of hubs; and
a pair of attachment bosses extending from the pair of hubs; and
a replacement strap assembly attached to the headgear assembly, wherein the replacement strap assembly comprises:
a pair of adapter rings, wherein each of the pair of adapter rings comprises an adapter ring aperture that is sized and configured to be placed over the corresponding attachment boss;
a replacement rear strap extending between a rear side of the pair of adapter rings; and
a replacement top strap extending between a top side of the pair of adapter rings.

2. The replacement rear strap assembly of claim 1, wherein each of the pair of attachment bosses is circle-shaped.

3. The replacement rear strap assembly of claim 2, wherein each of the adapter ring apertures is circle-shaped.

4. The replacement rear strap assembly of claim 1, wherein each of the pair of attachment bosses is D-shaped.

5. The replacement rear strap assembly of claim 2, wherein each of the adapter ring apertures is D-shaped.

6. The helmet of claim 1, wherein the replacement rear strap and replacement top strap comprise stretchable fabric.

7. A helmet comprising:
a headgear assembly comprising:
a front strap;
a top strap;
a pair of hubs; and
a pair of attachment bosses extending from the pair of hubs, wherein each of the attachment bosses is circle-shaped; and
a replacement rear strap assembly attached to the headgear assembly, wherein the replacement rear strap assembly comprises:
a pair of adapter rings, wherein each of the pair of adapter rings comprises an adapter ring aperture that is circle-shaped and is sized and configured to be placed over the corresponding attachment boss; and
a replacement rear strap extending between the pair of adapter rings.

8. The helmet of claim 7, wherein the replacement rear strap comprises stretchable fabric.

9. A helmet comprising:
a headgear assembly comprising:
a front strap;
a rear strap;
a pair of hubs; and
a pair of attachment bosses extending from the pair of hubs; and
a replacement top strap assembly attached to the headgear assembly, wherein the replacement top strap assembly comprises:
a pair of adapter rings, wherein each of the pair of adapter rings comprises an adapter ring aperture that is sized and configured to be placed over the corresponding attachment boss; and
a replacement top strap extending between the pair of adapter rings.

10. The replacement top strap assembly of claim 9, wherein each of the pair of attachment bosses is circle-shaped.

11. The replacement top strap assembly of claim 10, wherein each of the adapter ring apertures is circle-shaped.

12. The replacement top strap assembly of claim 9, wherein each of the pair of attachment bosses is D-shaped.

13. The replacement top strap assembly of claim 10, wherein each of the adapter ring apertures is D-shaped.

14. The helmet of claim 9, wherein the replacement top strap comprises stretchable fabric.

15. The helmet of claim 14, wherein the replacement top strap further comprises a first end and a second end that can be secured together using Velcro.
